(19) United States
(12) Patent Application Publication
(10) Pub. No.: US 2019/0307704 A1
(43) Pub. Date: Oct. 10, 2019

(54) METHOD FOR ENHANCED DELIVERY OF GENE BASED THERAPY AND VACCINATION USING ELECTROPORATION

(71) Applicant: Yuan Zhang, Saunderstown, RI (US)

(72) Inventor: Yuan Zhang, Saunderstown, RI (US)

(21) Appl. No.: 16/372,710

(22) Filed: Apr. 2, 2019

Related U.S. Application Data
(63) Continuation-in-part of application No. 15/979,874, filed on May 15, 2018.
Application No. 15/979,874 is a continuation of application No. 16/230,148, filed on Dec. 21, 2018.
(60) Provisional application No. 62/655,012, filed on Apr. 9, 2018.

(51) Int. Cl. A61K 9/51; A61K 39/39; A61K 39/00; A61K 39/12

(52) U.S. Cl. CPC A61K 9/51; A61K 39/39; A61K 39/0012; A61K 2039/55555; A61K 2039/51; A61K 2039/55516; A61K 39/12

(57) ABSTRACT

Embodiments of the present invention are generally related to a method of enhancing the transfection efficiency and immunological/pharmacological/therapeutic responses of concurrent gene-based vaccinations or therapies delivered by a wide range of non-viral particles, viral vectors or viral-biomaterials hybrid particles. In particular, embodiments of the present invention are directed to a method of electroporation which enhances the transfection efficiency of RNA replicon loaded lipid-based nanoparticles in-vivo as well as greatly improves immunogen-specific immune responses of such RNA-based vaccinations. This invention also applies to other gene-based vaccination and therapy using non-viral particles, viral based vectors or viral-biomaterials hybrid particles, in conjunction with the said electroporation regimen.

### CROSS-REFERENCE TO RELATED APPLICATIONS

The present application is a Continuation-in-Part (CIP) of U.S. application Ser. No. 15/979,874 entitled METHOD FOR ENHANCED NUCLEIC ACID TRANSFECTION USING A PEPTIDE filed on May 15, 2018 and U.S. application Ser. No. 16/230,148 entitled MULTIFUNCTIONAL NANOPARTICLE FOR VACCINATION filed on Dec. 21, 2019 which claim the benefit of U.S. Application No. 62/655,012 entitled METHOD FOR ENHANCED DELIVERY OF GENE BASED THERAPY AND VACCINATION USING ELECTROPORATION filed on Apr. 9, 2018.

### FIELD OF THE INVENTION

The present invention generally relates to a method of enhancing the transfection efficiency, biological and immunological effects of concurrent gene-based therapy or vaccinations delivered by non-viral particles or viral vectors. Specifically, the method entails an electroporation regimen to significantly enhance the transfection efficiency of gene loaded or nucleic acid loaded non-viral or viral based vectors in vivo as well as greatly improve immunogen-specific immune responses of such gene-based vaccinations.

### BACKGROUND OF THE INVENTION

Transfection is the process of artificially inserting foreign genetic material (e.g. DNA and RNA) into mammalian cells to produce genetically modified cells. The transfection process allows for the study of gene or gene products' function and regulation for use in a variety of applications such as producing transgenic organisms or providing gene therapy, while minimizing unintended effects such as cytotoxicity, off-target gene perturbation, or cell death.

RNA replicons (self-amplifying RNA) are derived from either positive- or negative-strand RNA viruses, from which at least one gene encoding essential structural protein has been deleted. They are regarded as “disabled” viruses unable to produce infectious progeny. RNA replicons contain the genes encoding alphavirus RNA replication machinery (non-structure proteins), but lack genes encoding the viral structural proteins required to make an infectious alphavirus particle. The structural protein genes are replaced with genes encoding proteins of interest for various medical applications.

One method of RNA replicon transfection involves the use of nanoparticles as carriers for nucleic acid. Nanoparticles are small (generally under 200 nanometers for desired medical application) objects that are either naturally or artificially produced. Despite their small size, they have a comparatively large surface for cellular adhesion compared to other particles for transfection, being small enough to cross the cell membrane into the cell with significant efficacy while simultaneously having the ability to localize within the cell at a high rate because of their large adhesive surface. Further, in addition to their enhanced propensity for transport into a cell, nanoparticles shield the incorporated nucleic acid from degradation by nucleases. As is well known in the art, nanoparticles can be synthesized using a wide variety of lipids, polymers, inorganic metals/ions, small molecules and the like biocompatible chemicals and materials.

The above materials can also be employed to formulate nucleic acids into microparticles, between 0.1 and 100 μm in size. The microparticles can be used as long-term depot formulations that can slowly release the entrapped cargos or the entrapped small nanovectors to the local tissue microenvironment for medical intervention.

In general, nanoparticle transfection is preferred over other transfection methods due to its sustained release characteristics and enhanced safety profile and biocompatibility over virus-mediated gene transfer. Nanoparticles are able to efficiently enter cells by exploiting the endocytosis pathway, followed by the entrapment in the acidic endosomal and lysosomal compartments for degradation. To prevent enzymatic degradation intracellularly, certain endosome-disrupting agents or rationally designed biomaterials (i.e. lipids, polymers) are usually incorporated into the nanoparticle carriers, in order to promote the therapeutic cargos (i.e. nucleic acids) entrapped in nanoparticles to release from endosomes/lysosomes to cytoplasm to exert their biological functions. Downstream events include transcribing (or as appropriate, translating) the nucleic acid for the desired result.

The intracellular delivery and transfection of nucleic acids, especially long nucleic acid molecules (e.g. RNA replicons), pose significant challenges for gene therapy. Thus there is a need in the field for improving the transfection efficiency of nucleic acid vaccines, and specifically RNA-based vaccines, using nanoparticles as non-viral delivery systems.

Virus-based vectors are also widely used in the gene transfection, such as Retroviruses, Lentivirus, Adenovirus, Herpes Simplex Virus (HSV), Adeno-Associated Viruses (AAV). The gene (or nucleic acid sequence) of interests can be packaged into viral vectors. The advances in vector technology and understandings of vector biology enhance viral-based gene therapy.

Electroporation is inducing cell membrane permeabilization by applying an external, high voltage, short-pulsed electrical field. Reversible electroporation refers to the temporary permeabilization and the membrane reseals after removing the field. If the membrane does not reseal, eventually followed by cell death, the procedure is irreversible electroporation. Electroporation can be used in applications in medicine, biotechnology and food industry, etc. The electric field properties which may affect the electroporation procedure are the pulse intensity, number of pulse, pulse duration and frequency, etc.

### SUMMARY OF THE INVENTION

Embodiments of the present invention are directed towards a method of enhancing the in vivo transfection efficacy and efficiency of RNA, using a lipid-based nanoparticle formulated to carry RNA replicons into the cell in combination with the use of an electroporation regimen. The invention seeks to increase the efficacy and efficiency of in vivo RNA replicon vaccination delivery as well as improve antigen-specific immune responses of RNA-based vaccinations. Embodiments of the invention may be modified for the in vivo transfection of any type of nucleic acid, including siRNA, miRNA, mRNA, cDNA, etc, as well as using any type of non-viral based particles and viral based vectors for the delivery of nucleic acids as mentioned.

Additionally, the viral vectors can be engineered, modified and functionalized with a wide variety of biomaterials to enhance the virus-mediated gene delivery, including but not limited to efficient delivery to specific cell types, improved tissue tropism, increased spread and migration to distant sites, modulate inflammation and immune responses, etc. These attributes of the viral-biomaterials hybrid particles may benefit gene transduction. Viral vectors can be combined with biomaterials either through encapsulation within the materials or immobilization onto a material surface, or other possible chemical and biological interactions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Accompanying this written specification is a collection of drawings of example embodiments of the present invention. One of ordinary skill in the art would appreciate that these are merely exemplary embodiments, and additional and alternative embodiments may exist and still be within the scope and spirit of the invention as described herein.

FIG. 1 shows a Cryogenic Transmission Electron Microscope (cryo-TEM) image of LNP-RNA, where the scale bar is 50 nm.

FIG. 2A is a graph demonstrating that intramuscular administration of an LNP-RNA formulation in conjunction with an electroporation regimen increases antigen expression levels in a subject over time after priming vaccination compared to when the subject is injected with the LNP-RNA formulation alone.

FIG. 2B is a graph demonstrating that intramuscular administration of an LNP-RNA formulation in conjunction with an electroporation regimen increases antigen expression levels in a subject over time after booster vaccination compared to when the subject is injected with the LNP-RNA formulation alone.

FIG. 3 is a graph demonstrating that intramuscular administration of an LNP-RNA formulation in conjunction with an electroporation regimen to a subject promotes an antigen-specific T-cell response after priming and booster immunizations. Particularly, injecting a subject with the LNP-RNA formulation in conjunction with the electroporation regimen enhances antigen-specific tetramer positive CD8⁺ T-cell clones in peripheral blood.

FIG. 4A shows a graph demonstrating that the tested LNP-RNA and electroporation regimen promotes an antigen specific T-cell response after priming and booster immunizations. Particularly, that injecting a subject with a vaccine containing the LNP-RNA formulation in conjunction with the electroporation regimen enhances T-cell effector function, as evidenced by the production of pro-inflammatory cytokine IFNγ in CD8⁺ cells in peripheral blood.

FIG. 4B shows a graph demonstrating that the tested LNP-RNA and electroporation regimen promotes an antigen specific T-cell response after priming and booster immunizations. Particularly, that injecting a subject with a vaccine containing the LNP-RNA formulation in conjunction with the electroporation regimen enhances T-cell effector function, as evidenced by the production of pro-inflammatory cytokine TNFα in CD8⁺ cells in peripheral blood.

FIG. 5A is an image of untreated muscle tissue.

FIG. 5B is an image of muscle tissue five hours after the muscle tissue was treated with an electroporation regimen.

FIG. 5C is an image of muscle tissue twenty-four hours after the muscle tissue was treated with an electroporation regimen.

### DETAILED DESCRIPTION OF THE INVENTION

The present disclosure is to be considered an exemplification of the invention, and is not intended to limit the invention to the specific embodiments illustrated by the figures or description below.

The terminology used herein is for the purpose of describing particular embodiments only and is not intended to be limiting of the invention. As used herein, the term “and/or” includes any and all combinations of one of more of the associated listed items. It will further be understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

In a preferred embodiment, the present invention discloses a method of enhancing the in vivo transfection efficacy and efficiency of RNA, using a lipid-based nanoparticle formulated to carry RNA replicons into the cell in combination with the use of an electroporation regimen. The invention seeks to increase the efficacy and efficiency of in vivo RNA-replicon vaccination delivery as well as improve antigen-specific immune responses of RNA-based vaccinations. The preferred embodiment can be modified so that it can be adapted for the in vivo transfection of any type of nucleic acid, including siRNA, miRNA, mRNA, cDNA, etc, as well as using any type of non-viral based particles and viral based vectors for the delivery of nucleic acids as mentioned

Additionally, the viral vectors can be engineered, modified and functionalized with a wide variety of biomaterials to enhance the virus-mediated gene delivery, including but not limited to efficient delivery to specific cell types, improved tissue tropism, increased spread and migration to distant sites, modulate inflammation and immune responses, etc. These attributes of the viral-biomaterials hybrid particles may benefit gene transduction. Viral vectors can be combined with biomaterials either through encapsulation within the materials or immobilization onto a material surface, or other possible chemical and biological interactions.

Traditional vaccination methods involving the artificial introduction of immunogens into mammalian cells to stimulate the immune system are known in the art as a means for artificially creating pathogen immunization in mammalian cells. These immunogens are generally infectious agents, or pathogens, that have been inactivated so that they will not cause disease. The traditional vaccination process facilitates a mammalian cell's creation of antibodies, or molecules specifically directed to counter the immunogens introduced into the body through a vaccination, which create a memory within the cells of the specific antigen (“acquired immunity”) and enable the vaccinated subject to rapidly and efficiently respond if the body ever becomes infected with the active pathogen.

In genetic vaccines, no immunogen is injected into the mammalian cell. Instead, only nucleic acids containing genetic information are introduced into the cell. Introduction of DNA or RNA into the cell provides mammalian cells with the genetic information required to produce the immunogen itself. The immunogen is then presented at the surface of a subset of cells and triggers the activation of the mammalian subject's immune system.

RNA replicon-based vaccines have several advantages and outperform conventional nucleic acid-based vaccines. First, the encoded viral non-structure proteins provide several rounds of intracellular replication, which increases mRNA templates and therefore enhances antigen provision beyond the quantity generated by conventional protein-based or mRNA-based vaccines. Importantly, comparing to mRNA, this RNA replicon construct triggers much longer gene expression, which lasts several weeks to months after one single injection. Second, as a result of the absence of structural proteins, alphavirus replicons have low intrinsic immunogenicity against the vector itself, allowing repeated immunizations with the same vector. Third, as the replication, transcription and translation of RNA replicons take place exclusively in the cytoplasm of target cells, the risk of genomic integration is eliminated and the RNA does not need to cross the nuclear membrane to be functional. The development of this non-transmissible RNA replicon therefore represents a significant advance towards safe vector vaccines. Fourth, RNA-based vaccine has self-adjuvating property. The viral RNA structure in replicons, in particular, can activate many innate immune pathways, such as toll-like receptors (TLRs) and stimulator of interferon genes (STING) pathways. Fifth, replicons can be genetically engineered to express multiple genes from the same construct, which offers the versatile capability of multi-antigen vaccination and minimizes potential viral immune escape following infections. Sixth, RNA can be synthesized in vitro in a cell-free manner, allowing large-scale, cost-effective production while avoiding complex manufacturing issues associated with recombinant proteins and viral vectors.

RNA-based genetic vaccines can be delivered into a subject by several routes and methods. Non-viral, needle-injection into skin and muscle tissue is most common. While methods for producing and injecting RNA into the cell are known in the art, the success rate of these injections has been lacking due to, in some circumstances, the genetic materials' inability to enter the cell membrane or to the cell's nucleus, and further, the genetic materials' inability to localize along the cell's nucleus to assist the nucleus in developing the desired immunity. To date, no method is known which uses electroporation to enhance the transfection efficiency of RNA nanoparticles in vivo and improve the antigen-specific immune responses of such RNA-based vaccines or improve therapeutic and pharmacological effects of any other forms of gene therapy.

The present invention provides a method for enhancing the transfection efficiency for in vivo delivery of nucleic acid, (e.g. RNA-replicons) into a cell using an electroporation regimen in conjunction with a lipid based nanoparticle (“LNP”) as the nucleic acid (e.g. RNA replicon) carrying vehicle. Numerous variations of the LNP are envisioned. In addition to LNP and RNA replicon, many types of non-viral particles and viral vectors can be employed to load many types of nucleic acids, and delivered in vivo in conjunction with the electroporation regimen.

Because of its physical nature, electroporation can be applied to practically any cell or tissue. The electroporation regimen disclosed herein subjects the cell membranes near the injection site to a high-voltage electric field, resulting in the membranes' temporary breakdown. Essentially, this breakdown formulates pores in the cell membranes that are large enough to allow macromolecules such as RNA replicons (nanoparticles) to enter the cell. During the time when the pores are open, RNA replicons (nanoparticles) are able to enter the cell. The use of an electroporation regimen in conjunction with the use of the LNP-RNA replicon carriers (nanoparticles) to introduce genetic material into the cell enhances the efficiency and efficacy of the transfection procedure. This allows a subject to be treated with a genetic vaccination that becomes more effective in vivo, and assists in the performance of gene therapy.

The LNP disclosed in the present invention is synthesized using 1,2-distearoyl-sn-glycero-3-phosphocholine (DSPC), cholesterol, 1,2-dioleoyl-3-trimethylammonium-propane (DOTAP), 1,2-distearoyl-sn-glycero-3-phosphoethanolamine-N-[methoxy(polyethylene glycol)-2000] (DSPE-PEG). An ethanol dilution process is used to produce the LNP formulation utilizing the following molar ratios of components: DSPC:cholesterol:DOTAP:DSPE-PEG 10:48:40:2 molar percent. The nitrogen (on DOTAP) to phosphate (on nucleic acid e.g. RNA) ratio (N:P) is 4:1 to approximately 16:1 depending on the size of entrapped RNA, though ideally 8:1. In a preferred method, equal volume of nucleic acid (e.g. RNA) in 100 mM citrate buffer (pH=6), wherein the nucleic acid concentration is 0.01-1 ug/uL depending on the nucleic acid treatment, dosage, and volume to be injected, is added into the lipid mixture in ethanol and mixed quickly using a pipette, and another equal volume of 100 mM citrate buffer (pH=6) is immediately added in the above mixture and mixed quickly using a pipette. The final mixture is incubated and shaken continuously at room temperature and after approximately 15 to 60 minutes, the mixture is dialyzed in sterile phosphate-buffered saline to produce the final LNP particles. In some embodiments, a modified version of the LNP is employed, wherein such version is a Lipid Polycation RNA (LPR). LPR may be prepared by a self-assembly method. For example, a cationic polypeptide may be complexed with RNAs via electrostatic interaction. The condensed LPR core may then be wrapped with either cationic or anionic liposomes via electrostatic interaction, followed by post insertion with DSPE-PEG. The cationic liposome for LPR may be prepared in the following manner: a 1:1 molar ratio combination of DOTAP and cholesterol dissolved in chloroform are vacuum dried. In some embodiments, an anionic liposome may be used for LPR preparation, wherein a 2:1:1 molar ratio combination of DOPA, DOPE, and cholesterol respectively dissolved in chloroform are vacuum dried. In both cases wherein LPR is prepared using cationic or anionic liposomes, the resulting thin lipid film after vacuuming may then be hydrated in 50° C. distilled water by vortexing for 1 minutes followed by sonicating in 50° C. water bath for 5˜10 minutes. The resulting liposomes are filtered through 200 nm and 100 nm membrane filters sequentially before use. In this example, the final total lipid concentration in liposomes is approximately 20 mM.

The electroporation method disclosed in the present invention comprises the injection of the LNPs carrying the desired RNA replicons into a subject, the placement of electrodes around the injection site, and the application of intermittent high-voltage electrical pulses, preferably 100 volts (V), to the injection site at a defined magnitude and length via the electrodes located around the injection site. In the preferred embodiment, the pulse length is 60 milliseconds (ms) and is applied in two continuous pulses.

More specifically, the present invention is accomplished by injecting nucleic acid-loaded LNP, e.g. replicon RNA-loaded LNP (LNP-RNA), into the subject's muscles (particularly, the quadricep muscle in mice) followed by a waiting period between thirty and sixty minutes. In alternative embodiments wherein the test subject is not mice, the LNP-RNA can be injected in other body parts (e.g. the human arm muscles). Conductive gel is then applied on the two electrodes and the electrodes will be tightly placed on the skin or muscle nearest or at the injection site, and the electroporating machine (BTX Harvard Apparatus ECM 830) is set to LV mode, 100V, with the pulse lengths set to 60 ms. The other way is to apply the above electroporation regimen first, and wait for thirty to sixty minutes before injecting the replicon RNA-loaded LNP into the electroporation site. The LNP and RNA replicon described here can be replaced to other types of non-viral particles or viral vectors and other types of nucleic acids for many gene-based therapy and vaccination.

The novelty of the current claimed invention lies in the dramatic increase of the transfection efficiency of RNA replicons in muscles or the injection site in vivo through the use of the electroporation regimen described above in conjunction with the use of the LNP to carry the RNA-replicons into the cell. Furthermore, the invention significantly increases the immunological and pharmacological responses in vivo using the said gene delivery regimen, integrating both non-viral synthetic nanoparticle delivery and a non-invasive electroporation regimen (or other physical methods).

In accordance with an embodiment of the present invention, provided is a method for delivering a vaccine composition to a tissue site in a subject comprising: (1) preparing the vaccine composition by modifying a lipid based nanoparticle to load nucleic acid; (2) administering the vaccine composition to the tissue site in the subject; (3) placing electrodes around the tissue site; and (4) applying intermittent high-voltage electrical pulses to the tissue site at a defined magnitude and length via the electrodes located around the tissue site. In some embodiments, the nucleic acid is RNA. In some embodiments, the high-voltage electrical pulses are 100V pulses. In some embodiments, the pulse length is 60 ms. In some embodiments, the electric current is applied in two continuous 60 ms pulses.

In accordance with an embodiment of the present invention, provided is a method for delivering a vaccine composition to a tissue site in a subject wherein electrical pulses are first applied to the subject followed by injection of a vaccine comprising modified lipid based nanoparticles carrying nucleic acids. In some embodiments the method comprises: (1) placing electrodes around the tissue site; (2) applying intermittent high-voltage electrical pulses to the tissue site at a defined magnitude and length via the electrodes located around the tissue site; (3) preparing the vaccine composition by modifying a lipid based nanoparticle to load nucleic acid; and (4) administering the vaccine composition to the tissue site in the subject after the intermittent high-voltage electrical pulses have been applied. In some embodiments, the nucleic acid is RNA. In some embodiments, the high-voltage electrical pulses are 100V pulses. In some embodiments, the pulse length is 60 ms. In some embodiments, the electric current is applied in two continuous 60 ms pulses.

In accordance with an embodiment of the present invention, provided is a system for promoting cell transfection in a subject, the system comprising a means for administering a nucleic acid formulation to the subject, and a high voltage electric current applicator for applying an electric current to the subject. In some embodiments, the nucleic acid formulation is a lipid based nanoparticle loading nucleic acid. In some embodiments, the nucleic acid is RNA. In some embodiments, the electric current is 100V. In some embodiments, the electric current is applied intermittently. In some embodiments, the electric current is applied for a length of 60 ms. In some embodiments, the electric current is applied in two continuous 60 ms pulses.

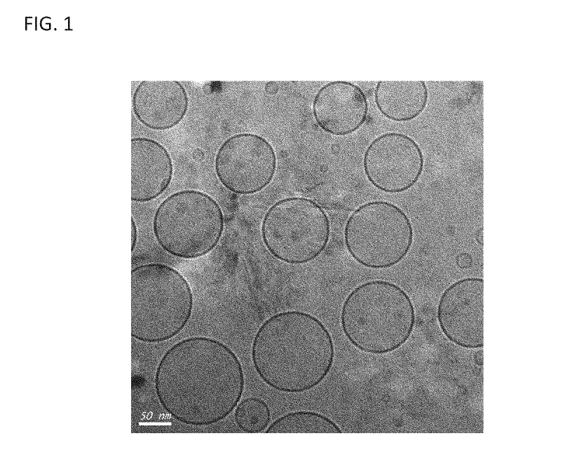

Turning now to FIGS. 1-5, various aspects of the method for enhanced delivery of gene based therapy and vaccination using electroporation in accordance with an embodiment of the present invention are shown.

FIG. 1 shows a cryogenic transmission electron microscope (cryo-TEM) image of LNP loaded with RNA (LNP-RNA) where the scale bar is 50 nm.

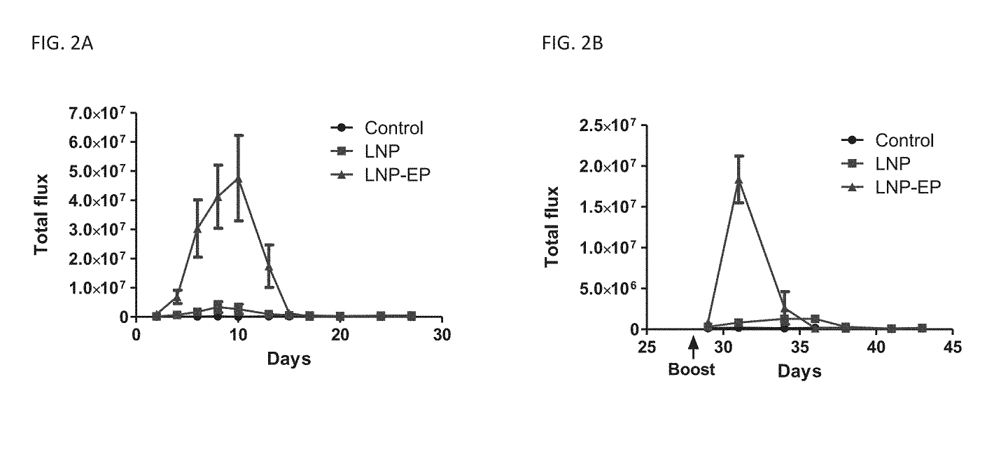

FIG. 2 is a display of graphs comparing antigen expression after an LNP vaccination regimen without electroporation and an LNP vaccination regimen incorporating electroporation. To collect the data in FIGS. 2(A-B), albino C57BL/6 mice were vaccinated with LNP containing 1 μg replicon RNA encoding SIVgag-Fluc2 model antigen. The subjects were primed with the LNP vaccination regimen on day 0, and boosted with the vaccine on day 28. Some of the tested subjects underwent an electroporation treatment, wherein electroporation pulses were applied to the injection site, in this case muscle tissue, approximately thirty to sixty minutes after the injection was administered, while the rest of the subject were solely administered with the LNP-RNA formulation alone. As shown in FIGS. 2(A-B), compared to the subjects injected with the LNP-RNA formulation alone, the subjects injected with the LNP-RNA formulation in conjunction with undergoing an electroporation regimen thereafter experienced increased antigen expression levels at the muscle injection site after priming on day 0 (FIG. 2A) and after boosting on day 28 (FIG. 2B).

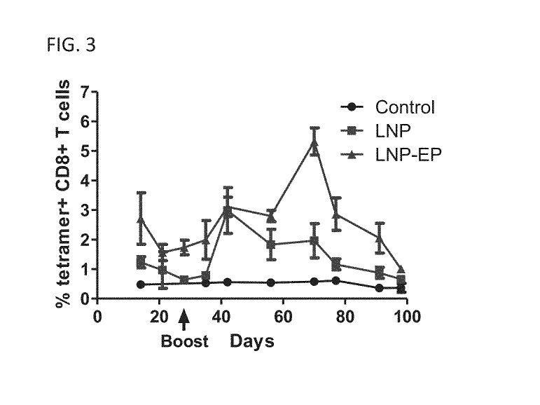

FIG. 3 is a display of graphs comparing the presence of antigen-specific tetramer positive CD8⁺ T-cell clones in peripheral blood after an LNP vaccination regimen without electroporation and an LNP vaccination regimen incorporating electroporation. To collect the data in FIGS. 3(A-B), C57BL/6 mice were vaccinated with LNP containing 1 μg replicon RNA encoding SlVgag-Fluc2 model antigen. The subjects were primed with the LNP vaccination regimen on day 0, and boosted with the vaccine on day 28. The peripheral blood of the tested subjects was collected and evaluated to test the efficacy of the LNP-RNA vaccination in conjunction with the electroporation regimen compared to the LNP-RNA vaccination alone. As shown in FIG. 3, the subjects injected with the LNP-RNA formulation in conjunction with undergoing an electroporation regimen thereafter exhibited an increase in and persistence of produced tetramer positive CD8⁺ T-cells, suggesting significant clonal expansion of antigen-specific CD8⁺ T-cells. Furthermore, as shown by the graph in FIG. 3, the antigen-specific T-cell clone persisted in peripheral blood for more than three months after a single boost on day 28.

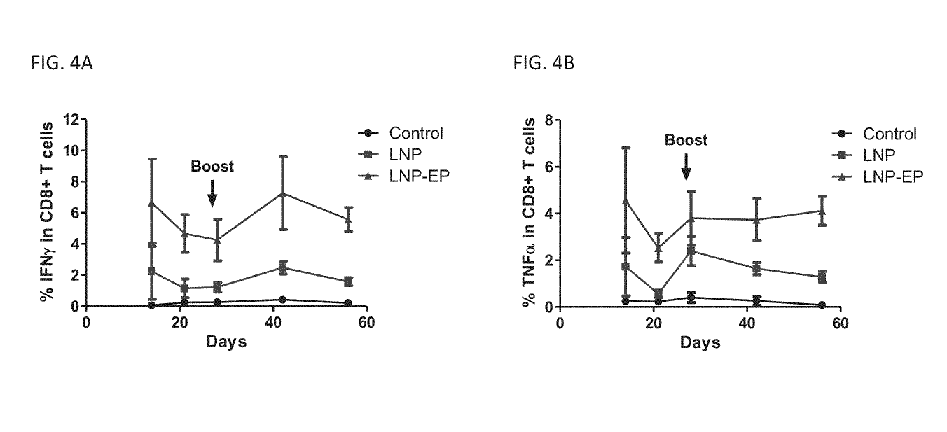

FIG. 4A shows a graph demonstrating that the tested LNP-RNA and electroporation regimen promotes an antigen specific T-cell response after immunization. To collect the data in FIGS. 4(A-B), C57BL/6 mice were vaccinated with LNP containing 1 μg replicon RNA encoding SlVgag-Fluc2 model antigen. The subjects were primed with the LNP vaccination regimen on day 0, and boosted with the vaccine on day 28. Peripheral blood was drawn weekly and subjected to ex vivo stimulation with a cognate peptide to analyze the antigen-specific T-cell effector functions by intracellularly staining pro-inflammatory cytokines IFNγ and TNFα. As shown in FIG. 4A, injecting a subject with a vaccine containing the LNP-RNA formulation in conjunction with the electroporation regimen enhances T-cell effector function, as evidenced by the production of pro-inflammatory cytokine IFNγ in CD8⁺ cells in peripheral blood. As shown in FIG. 4B, injecting a subject with a vaccine containing the LNP-RNA formulation in conjunction with the electroporation regimen enhances T-cell effector function, as evidenced by the production of pro-inflammatory cytokine TNFα in CD8⁺ cells in peripheral blood. Furthermore, as shown by the graph in FIGS. 4(A-B), the cytokine production persisted at a high level for at least two months after a single boost on day 28.

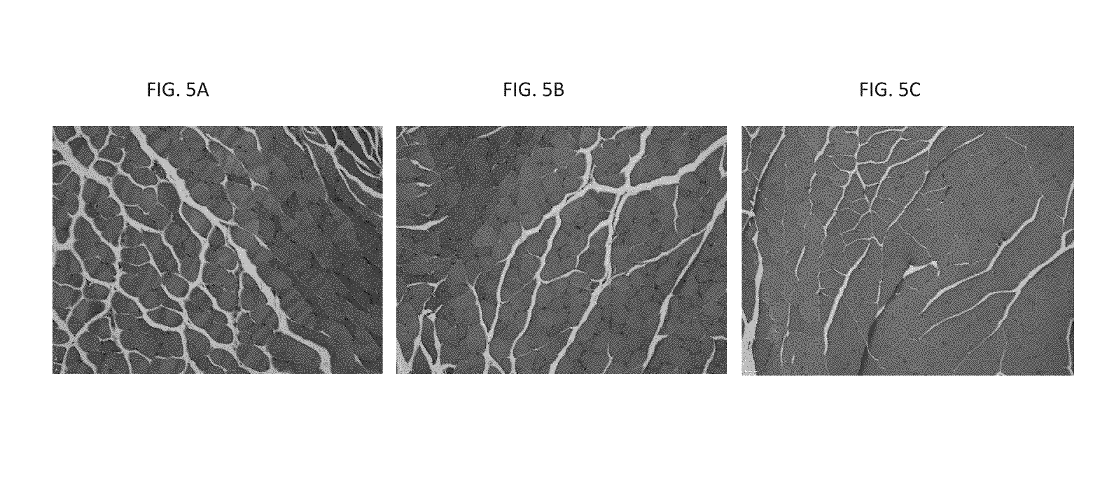

FIG. 5(A-C) are images showing untreated muscle tissues and muscle tissues treated with an electroporation regimen. Specifically, FIG. 5A is an image of untreated muscle tissue, FIG. 5B is an image of muscle tissue five hours after the muscle tissue was treated with an electroporation regimen, and FIG. 5C is an image of muscle tissue twenty-four hours after the muscle tissue was treated with an electroporation regimen. As evidenced by the three images in FIG. 5, the electroporation regimen did not induce any muscle damage to the test site.

While multiple embodiments are disclosed, still other embodiments of the present invention will become apparent to those skilled in the art from this detailed description. There may be aspects of this invention that may be practiced without the implementation of some features as they are described. It should be understood that some details have not been described in detail in order to not unnecessarily obscure the focus of the invention. The invention is capable of myriad modifications in various obvious aspects, all without departing from the spirit and scope of the present invention. Accordingly, the drawings and descriptions are to be regarded as illustrative rather than restrictive in nature.

1. A method for delivering a vaccine composition in vivo to a tissue site in a subject comprising:
preparing the vaccine composition by modifying a nanoparticle to load nucleic acid;
administering the vaccine composition to the tissue site in the subject;
placing electrodes around the tissue site; and
applying intermittent high-voltage electrical pulses to the tissue site at a defined magnitude and length via the electrodes positioned around the tissue site.

2. The method for delivering a vaccine composition to a tissue site in a subject in accordance with claim 1, wherein the nucleic acid is RNA.

3. The method for delivering a vaccine composition to a tissue site in a subject in accordance with claim 1, wherein the high-voltage electrical pulses are 100V in magnitude.

4. The method for delivering a vaccine composition to a tissue site in a subject in accordance with claim 1, wherein the pulse length is 60 ms.

5. The method for delivering a vaccine composition to a tissue site in a subject in accordance with claim 1, wherein the pulse length is 60 ms and is applied in two successive pulses.

6. The method for delivering a vaccine composition to a tissue site in a subject in accordance with claim 1, wherein the tissue is muscle tissue.

7. The method for delivering a vaccine composition to a tissue site in a subject in accordance with claim 1, wherein the time interval between administering the vaccine composition to the tissue site in the subject and applying intermittent high-voltage electrical pulses to the tissue site is 30 to 60 minutes.

8. A method for delivering a vaccine composition in vivo to a muscle tissue site in a subject comprising:
preparing the vaccine composition by modifying a nanoparticle to load nucleic acid;
administering the vaccine composition to the muscle tissue site in the subject;
placing electrodes around the muscle tissue site; and
applying intermittent high-voltage electrical pulses to the muscle tissue site at a defined magnitude and length via the electrodes positioned around the muscle tissue site.

9. A method for delivering a vaccine composition to a muscle tissue site in a subject in accordance with claim 8, wherein the nucleic acid is RNA.

10. The method for delivering a vaccine composition to a muscle tissue site in a subject in accordance with claim 8, wherein the high-voltage electrical pulses are 100V pulses.

11. The method for delivering a vaccine composition to a muscle tissue site in a subject in accordance with claim 8, wherein the pulse length is 60 ms.

12. The method for delivering a vaccine composition to a muscle tissue site in a subject in accordance with claim 8, wherein the pulse length is 60 ms and is applied in two continuous pulses.

13. The method for delivering a vaccine composition to a muscle tissue site in a subject in accordance with claim 8, wherein the time interval between administering the vaccine composition to the tissue site in the subject and applying intermittent high-voltage electrical pulses to the tissue site is 30 to 60 minutes.

14. A system for promoting cell in vivo transfection in a subject, the system comprising:
a means for administering a nucleic acid formulation to the subject; and
a high voltage electric current applicator for applying an electric current to the subject.

15. The system for promoting cell transfection in a subject in accordance with claim 14, wherein the nucleic acid formulation is a lipid based nanoparticle loading nucleic acid.

16. The system for promoting cell transfection in a subject in accordance with claim 14, wherein the nucleic acid formulation is a lipid based nanoparticle loading RNA.

17. The system for promoting cell transfection in a subject in accordance with claim 14, wherein the magnitude of the electrical pulses is 100V.

18. The system for promoting cell transfection in a subject in accordance with claim 14, wherein the electric current is applied intermittently.

19. The system for promoting cell transfection in a subject in accordance with claim 14, wherein the electric current is applied for a length of 60 ms.

20. The system for promoting cell transfection in a subject in accordance with claim 14, wherein the electric current is applied in two continuous 60 ms pulses.

21. The system for promoting cell transfection in a subject in accordance with claim 14, wherein the time interval between the application of high-voltage electrical pulses and the administration of vaccine composition to the tissue site is 30 to 60 minutes.
